(19) United States
(12) Patent Application Publication
(10) Pub. No.: US 2020/0352517 A1
(43) Pub. Date: Nov. 12, 2020

(54) METHOD OF PREPROCESSING NEAR INFRARED (NIR) SPECTROSCOPY DATA FOR NON-INVASIVE GLUCOSE MONITORING AND APPARATUS THEREOF

(71) Applicant: SAMSUNG ELECTRONICS CO., LTD., Suwon-si (KR)

(72) Inventors: Sujit Jos, Kerala (IN); Kiran Bynam, Jalahalli (IN); Rahul Arora, Uttar Pradesh (IN); Ibrahim Abdul Majeed, Kerala (IN); So Young Lee, Daejeon (KR)

(73) Assignee: SAMSUNG ELECTRONICS CO., LTD., Suwon-si (KR)

(21) Appl. No.: 16/869,791

(22) Filed: May 8, 2020

(30) Foreign Application Priority Data
May 8, 2019 (IN) 201941018443
Feb. 28, 2020 (KR) 10-2020-0025063

(51) Int. Cl. A61B 5/00; G01N 21/359; G01N 21/3577; A61B 5/145; A61B 5/1455

(52) U.S. Cl. CPC A61B 5/7203; G01N 21/359; G01N 21/3577; A61B 5/725; A61B 5/1455; A61B 5/7246; A61B 5/726; A61B 5/14532

(57) ABSTRACT

The present disclosure relates to a method and system for preprocessing near infrared (NIR) spectroscopy data for non-invasive monitoring of blood glucose. In accordance with an embodiment, the method receiving the NIR spectroscopy data from a subject; performing a scatter correction on the NIR spectroscopy data to obtain scatter corrected NIR spectra; removing interference from the scatter corrected NIR spectra to obtain glucose spectra; removing noise from the glucose spectra to obtain noise removed glucose spectra; obtaining noise removed NIR glucose data as a set of noise removed glucose spectra corresponding to a plurality of reference glucose values; removing drift from the noise removed NIR glucose data to obtain preprocessed NIR glucose data; and obtaining a set of global features from the preprocessed NIR glucose data for non-invasive monitoring of blood glucose of the subject.

### CROSS-REFERENCE TO RELATED APPLICATION(S)

This application is based on and claims priority under 35 U.S.C. § 119 to Indian Patent Application No. 201941018443, filed on May 8, 2019, in the Indian Intellectual Property Office, and Korean Patent Application No. 10-2020-0025063, filed on Feb. 28, 2020 in the Korean Intellectual Property Office, the disclosures of which are incorporated by reference herein in their entireties.

### BACKGROUND

### 1. Field

The present disclosure generally relates to non-invasive monitoring of blood glucose of a subject, and particularly relates to a method and system for preprocessing near infrared (NIR) spectroscopy data for the non-invasive monitoring of blood glucose.

### 2. Description of Related Art

With the advent of technology, non-invasive monitoring of blood glucose has gained wide interest. The objective of non-invasive monitoring of blood glucose is to provide a non-invasive technique of monitoring of blood glucose such as without the need for finger pricking, obtaining a drop of blood, or using a test strip. Spectroscopy-based non-invasive techniques are considered promising technologies. A spectroscopy-based non-invasive technique includes passing a band of radiation through a vascular region of the body and determining concentrations of glucose by analyzing the reflected or transmitted spectrum. Such a non-invasive technique is especially useful to patients for whom glucose values are monitored mandatorily several times a day. Popular spectroscopy-based non-invasive techniques include near infrared (NIR) spectroscopy, mid infrared (MIR) spectroscopy, and Raman spectroscopy. These methods primarily differ in the utilized wavelengths of electromagnetic (EM) spectrum for analysis of interstitial fluid tissue.

Presently, the NIR spectroscopy-based non-invasive technique is one of the most commonly tried methods for prediction of blood glucose. NIR spectroscopy is associated with an EM spectrum in the range of 750-2500 nanometers (nm). The absorption of NIR light in bio-fluids is caused by the presence of C—H, O—H and N—H bonds, which absorb light in combination and overtone regions. Glucose primarily absorbs NIR light in two distinct regions, namely, (a) a first overtone region that is between 1500 nm to 1800 nm, and (b) a second combination band region. In this context, the NIR spectra are described as follows using the Beer-Lambert's law. Beer-Lambert's law is the linear relationship between absorbance and concentration of an absorbing species. The Beer-Lambert's law is usually expressed as:

$${A}_{λ}^{g} = - log ( \frac{I}{{I}_{0}} ) = {ϵ}_{λ}^{g} {c}_{g} d \qquad ( 1 )$$

As shown above, A_{λ}^{g} is the wavelength dependent absorbance of glucose, and ∈_{λ}^{g} is the wavelength dependent molar absorptivity coefficient of glucose with units M⁻¹cm⁻¹. c₉ is the glucose concentration and d is the path length. I₀ is the intensity of the original incident NIR light and I is the light intensity after it passes through the sample. The spectra A_{λ}^{g} are referred to as the glucose spectra corresponding to the glucose concentration c_{g}. The glucose concentration c_{g} may also be referred to as a “glucose value” elsewhere herein.

However, employing NIR spectroscopy for glucose prediction has various challenges. First, the absorption coefficient and concentration in the range of 750-2500 nm are such that the glucose signal constitutes about only 1 part in 100,000, whereas the major contribution is due to water followed by other blood-compounds such as hemoglobin, proteins, and fat. Therefore, the stronger NIR spectra of these compounds overlap with the weak spectral bands of glucose. Second, the NIR measurements are sensitive to a variety of environmental effects such as temperature, humidity, ambient lighting, and device dependent drift. Third, the NIR measurement varies with test subject bio-profile. These effects manifest themselves as drift and noise, and affect the feature extraction and consequently the prediction results based on the NIR spectroscopy data.

Various solutions are present that overcome above-mentioned deficiencies. In one solution, a spectrometer measures the near-infrared spectrum of a subject's tissue. An analyzer processes the spectral measurement and extracts features relevant to outlier detection and glucose measurement. The analyzer applies a model to the processed spectral measurement and/or the extracted features to obtain a glucose measurement. In another solution, influence of measurement conditions, human body physiological backgrounds, and the like, on blood glucose concentration measurement can be comprehensively taken into account, according to physiological data, spectral data, blood glucose concentration truth value, and non-blood-glucose concentration data of many sample testers, based on a multivariate calibration method. A blood glucose concentration prediction model based on “M+N” theory is established. Through the model, the prediction of blood glucose concentration is performed. However, these solutions do not address spectral correction and filtering; and consistent features are not obtained for regression.

In another solution, measurement of blood glucose is performed using a portion of the IR spectrum, which contains the NIR water absorption peaks. Electromagnetic radiation of a wavelength is transmitted through the skin to the measurement region, for example, a blood vessel. The collected light is analyzed and compared against a stored reference calibration curve to calculate blood glucose concentration. However, in this solution, the spectral interference caused by the background non-analyte is high, thus reducing SNR and accuracy. Further, the solution uses a reference calibration curve which varies from person to person and hence universality is not guaranteed. In addition, the solution assumes that the background interference is common for all range of the near-infrared region, thereby further reducing the accuracy.

Thus, there exists a need for a solution to overcome above-mentioned deficiencies.

### SUMMARY

This summary is provided to introduce a selection of concepts in a simplified format that are further described in the detailed description of the present disclosure. This summary is not intended to identify key or essential inventive concepts of the claimed subject matter, nor is it intended for determining the scope of the claimed subject matter. In accordance with the purposes of the disclosure, the present disclosure as embodied and broadly described herein, describes method and system for preprocessing near infrared (NIR) spectroscopy data for non-invasive monitoring of blood glucose.

In accordance with an aspect of the disclosure, a method for preprocessing near infrared (NIR) spectroscopy data for non-invasive monitoring of blood glucose may include receiving the NIR spectroscopy data from a subject; performing a scatter correction on the NIR spectroscopy data to obtain scatter corrected NIR spectra; removing interference from the scatter corrected NIR spectra to obtain glucose spectra; removing noise from the glucose spectra to obtain noise removed glucose spectra; obtaining noise removed NIR glucose data as a set of noise removed glucose spectra corresponding to a plurality of reference glucose values; removing drift from the noise removed NIR glucose data to obtain preprocessed NIR glucose data; and obtaining a set of global features from the preprocessed NIR glucose data for non-invasive monitoring of blood glucose of the subject.

According to an aspect of the disclosure, a system for preprocessing near infrared (NIR) spectroscopy data for non-invasive monitoring of blood glucose may include a memory configured to store instructions; and a processor configured to execute the instructions to perform a scatter correction on NIR spectroscopy data from a subject to obtain scatter corrected NIR spectra; remove interference from the scatter corrected NIR spectra to obtain glucose spectra; remove noise from the glucose spectra to obtain noise removed glucose spectra; obtain noise removed NIR glucose data as a set of noise removed glucose spectra corresponding to plurality of reference glucose values; remove drift from the noise removed glucose spectra to obtain preprocessed NIR glucose data; and obtain a set of global features from the preprocessed NIR glucose data for non-invasive monitoring of the blood glucose of the subject.

The embodiments of the present disclosure provide preprocessing of the NIR spectroscopy data such that the effects of noise and drift are removed from the NIR spectroscopy data prior to predicting blood glucose levels. Further, the preprocessing of the NIR spectroscopy data includes scatter correction and noise removal along with drift correction to obtain high quality data. This results in improvement in the quality of the data, resulting in better prediction accuracy of the blood glucose value using standard machine learning methods. Further, the preprocessing of the NIR spectroscopy data obtains global features that exhibit high correlation with the reference glucose values and are therefore universal, i.e. the features are common across all test subjects.

These and additional aspects and advantages will be more clearly understood from the following detailed description taken in conjunction with the accompanying drawings and claims.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 illustrates a schematic block diagram of a system for preprocessing near infrared (NIR) spectroscopy data for non-invasive monitoring of blood glucose, according to an embodiment;

FIG. 2 illustrates an example graph indicative of the preprocessing of NIR spectroscopy data, according to an embodiment;

FIG. 3 illustrates an example graph indicative of the preprocessing of NIR spectroscopy data, according to an embodiment;

FIG. 4 illustrates an example graph indicative of the preprocessing of NIR spectroscopy data, according to an embodiment;

FIG. 5 illustrates an example graph indicative of the preprocessing of NIR spectroscopy data, according to an embodiment;

FIG. 6 illustrates an example graph indicative of the preprocessing of NIR spectroscopy data, according to an embodiment;

FIG. 7 illustrates an example graph indicative of the preprocessing of NIR spectroscopy data, according to an embodiment;

FIG. 8 illustrates an example graph indicative of the preprocessing of NIR spectroscopy data, according to an embodiment;

FIG. 9 illustrates a flow diagram of a method for preprocessing near infrared (NIR) spectroscopy data for non-invasive monitoring of blood glucose according to an embodiment;

FIG. 10 illustrates a flow diagram of a method for preprocessing NIR spectroscopy data for non-invasive monitoring of blood glucose according to an embodiment;

FIG. 11 illustrates a flow diagram of a method for preprocessing NIR spectroscopy data for non-invasive monitoring of blood glucose according to an embodiment;

FIG. 12 illustrates a flow diagram of a method for preprocessing NIR spectroscopy data for non-invasive monitoring of blood glucose according to an embodiment;

FIG. 13 illustrates a flow diagram of a method for preprocessing near infrared (NIR) spectroscopy data for non-invasive monitoring of blood glucose according to an embodiment;

FIG. 14 illustrates a flow diagram of a method for preprocessing NIR spectroscopy data for non-invasive monitoring of blood glucose according to an embodiment; and

FIG. 15 illustrates a flow diagram of a method for preprocessing NIR spectroscopy data for non-invasive monitoring of blood glucose according to an embodiment.

Further, skilled artisans will appreciate that elements in the drawings are illustrated for simplicity and may not have been necessarily drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION

For the purpose of promoting an understanding of the principles of the disclosure, reference will now be made to the embodiments illustrated in the drawings and specific language will be used to describe the same. It will nevertheless be understood that no limitation of the scope of the disclosure is thereby intended, such alterations and further modifications in the illustrated system, and such further applications of the principles of the disclosure as illustrated therein being contemplated as would normally occur to one skilled in the art to which the disclosure relates. Unless otherwise defined, all technical and scientific terms used herein have the same meaning as commonly understood by one of ordinary skill in the art to which this disclosure belongs. The system, methods, and examples provided herein are illustrative only and are not intended to be limiting. Embodiments of the present disclosure will be described below in detail with reference to the accompanying drawings.

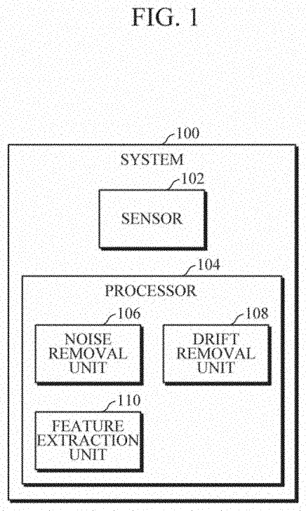

FIG. 1 illustrates a schematic block diagram of a system 100 for preprocessing near infrared (NIR) spectroscopy data for non-invasive monitoring of blood glucose, according to an embodiment. In an implementation, the system 100 can be a standalone device. In another implementation, the system 100 can be integrated with a mobile device such as a smartphone, and a wearable device such as a smart watch, a fitness band, an arm band, etc.

In accordance with an embodiment, the system 100 includes a sensor102 and a processor 104. The sensor 102 and the processor 104 are communicatively coupled with each other via a bus (not shown). The sensor102 and the processor 104 may be further communicatively coupled with other components such as a memory. In an implementation, the system 100 can be a standalone device. In another implementation, the system 100 can be integrated with mobile devices and wearable devices. Further, the processor 104 may be implemented in hardware, software, or by a combination thereof.

The bus may include a component that permits communication among the components of system 100. The processor 104 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component.

The processor 104 may include one or more processors capable of being programmed to perform a function. A memory may include a random access memory (RAM), a read only memory (ROM), and/or another type of dynamic or static storage device (e.g., a flash memory, a magnetic memory, and/or an optical memory) that stores information and/or instructions for use by the processor 104. A storage component may store information and/or software related to the operation and use of the system 100. For example, a storage component may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

The sensor 102 may include an input component that permits the system 100 to receive information, such as via user input (e.g., a touch screen display, a keyboard, a keypad, a mouse, a button, a switch, and/or a microphone). Additionally, or alternatively, the sensor 102 may include a sensor for sensing information (e.g., a transmitter, an emitter, a detector, a light source, a global positioning system (GPS) component, an accelerometer, a gyroscope, and/or an actuator). The system 100 may include an output component (now shown) that provides output information from the system 100 (e.g., a display, a speaker, and/or one or more light-emitting diodes (LEDs)).

The sensor 102 may include a communication interface such as a transceiver-like component (e.g., a transceiver and/or a separate receiver and transmitter) that enables the system 100 to communicate with other devices, such as via a wired connection, a wireless connection, or a combination of wired and wireless connections. The communication interface may permit the system 100 to receive information from another device and/or provide information to another device. For example, the communication interface may include an Ethernet interface, an optical interface, a coaxial interface, an infrared interface, a radio frequency (RF) interface, a universal serial bus (USB) interface, a Wi-Fi interface, a cellular network interface, or the like.

The system 100 may perform one or more processes described herein. The system 100 may perform these processes in response to the processor 104 executing software instructions stored by a non-transitory computer-readable medium, such as a memory and/or a storage component. A computer-readable medium is defined herein as a non-transitory memory device. A memory device includes memory space within a single physical storage device or memory space spread across multiple physical storage devices.

Software instructions may be read into the memory and/or storage component from another computer-readable medium or from another device via communication interface. When executed, software instructions stored in the memory and/or storage component may cause the processor 104 to perform one or more processes described herein.

Additionally, or alternatively, hardwired circuitry may be used in place of or in combination with software instructions to perform one or more processes described herein. Thus, implementations described herein are not limited to any specific combination of hardware circuitry and software.

In accordance with an embodiment, the sensor 102 may receive the NIR spectroscopy data from a subject. The NIR spectroscopy data can be obtained by transmitting an NIR radiation from a transmitter (not shown) of the system 100 through the skin of the subject to a measurement region, for example, a blood vessel, and collecting or receiving reflected light as NIR spectroscopy data. The measurement region can be any body part of the subject such as palm of hand of the subject, finger of the subject, wrist of the subject, upper arm of the subject, etc. The transmitter may include a monochromator that produces a light beam in the NIR wavelength band, i.e., 750 nanometer (nm) to 2500 nm band, from a light emitted from a light source and the light beam is directed onto the skin of the subject. The sensor 102 may include a detector to detect the light that is reflected off the skin of the subject. The transmitter and the detector may be located in the standalone device or the mobile device or the wearable device at suitable positions to perform the foregoing functions.

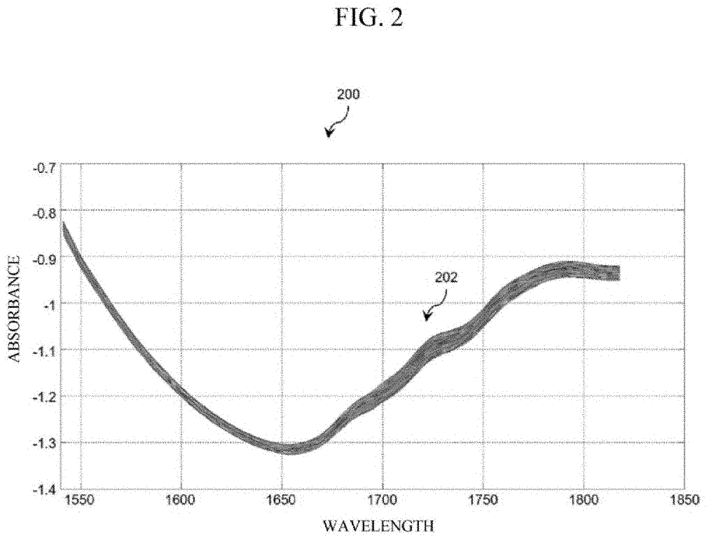

The NIR spectroscopy data comprises spectra of a plurality of interfering components and glucose spectra. The plurality of interfering components includes known components such as water, other blood-compounds like hemoglobin, proteins and fat, temperature, hydrogen, bonding effects, scatter correction, refractive index correction, and depth of penetration, and unknown components. FIG. 2 illustrates an example of plurality of NIR spectroscopy data 200 corresponding to different reference glucose values with the X-axis representing wavelength (e.g., measured in nanometers) and the Y-axis representing absorbance. Each of the curves 202 represent the NIR spectroscopy data obtained from the subject comprising of spectra of the plurality of interfering components and the glucose spectra.

Based on receiving the NIR spectroscopy data, the processor 104 preprocesses near NIR spectroscopy data for the non-invasive monitoring of blood glucose. The processor (104) may obtain the noise removed glucose spectra from the NIR spectroscopy data. The processor 104 may remove drift from the noise removed glucose spectra to obtain a preprocessed NIR glucose data. The processor 104 may obtain a set of global features from the preprocessed NIR glucose data for non-invasive monitoring of the blood glucose of the subject.

In an implementation, a single unit i.e., the processor 104 performs all the aforementioned steps. In another implementation, the processor 104 may include different units/modules that individually perform aforementioned steps. For example, the processor 104 may include a noise removal unit 106 to obtain the noise removed glucose spectra from the NIR spectroscopy data. The processor 104 may, for example, include a drift removal unit 108 to remove drift from the noise removed glucose spectra to obtain preprocessed NIR glucose data. The processor 104 may, for example, include a feature extraction unit 110 to obtain a set of global features from the preprocessed NIR glucose data for non-invasive monitoring of the blood glucose of the subject.

In accordance with an embodiment, the processor 104 may perform a scatter correction on the NIR spectroscopy data to obtain a scatter corrected NIR spectra. The processor 104 may subtract a mean of the NIR spectroscopy data from each component of the NIR spectroscopy data to obtain a zero-mean NIR spectroscopy data. The processor 104 may divide the zero-mean NIR spectroscopy data with a numerical constant to obtain the scatter corrected NIR spectroscopy data. In an implementation, the scatter correction is performed by applying modified Standard Normal Variate (SNV) to the NIR spectroscopy data for correcting the undesired scattering effect. The SNV corrected NIR spectra may be represented by the equation (1) shown below.

$${x}_{corrected} = \frac{{x}_{observed} - μ}{σ} \qquad ( 1 )$$

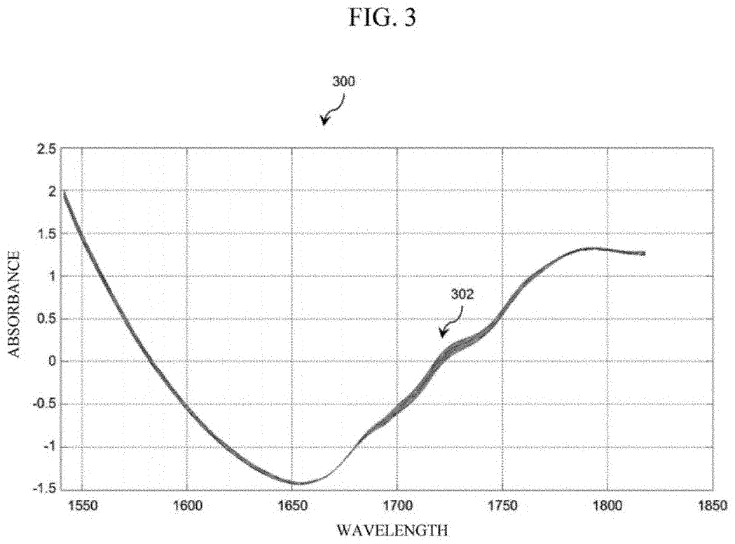

As shown above, μ is the mean of the signal x_{observed} and a is the standard deviation of the observed spectra. A numerical constant c is used instead of standard deviation σ to obtain the SNV corrected NIR spectra in accordance with the embodiment. FIG. 3 illustrates an example of plurality of scatter corrected NIR spectra 300 corresponding to different reference glucose values with the X-axis representing wavelength (e.g., measured in nanometers) and the Y-axis representing absorbance. Each of the curves 302 represent the scatter corrected NIR spectra obtaining by applying the modified SNV to the NIR spectroscopy data represented as curves 202 in FIG. 2.

Thereafter, the processor 104 may remove interference from the scatter corrected NIR spectra to obtain glucose spectra. The processor 104 may apply Extended Multiplicative scattering correction (EMSC) to the scatter corrected NIR spectra to obtain the glucose spectra. The scatter corrected NIR spectra comprises spectra of the plurality of interfering components and the glucose spectra. As such the overall absorption at wavelength λ is given by the equation (2) shown below.

A_{λ}=∈_{λ}^{g}c_{g}d+∈_{λ}¹c¹d+∈_{λ}²c²d+ . . . +∈ₙcⁿd (2)

As shown above, c¹, c², . . . cⁿ are concentrations of the interfering components. By applying the EMSC, the processor 104 may remove spectra of the plurality of interfering components from the scatter corrected NIR spectra to obtain the glucose spectra.

The processor 104 may then remove noise from the glucose spectra to obtain noise removed glucose spectra. The processor 104 may apply predefined spectral filter to the glucose spectra to obtain the noise removed glucose spectra. In an implementation, the predefined spectral filter is a Norris-Williams filter. Spectral derivatives are employed for combating the additive and multiplicative effects in a signal. Spectral derivatives result in nose inflation due to the differencing operation. The Norris-Williams (NW) filter implements the spectral filtering by computing the first derivative while controlling noise inflation. Accordingly, the spectral derivatives are computed using the following steps:

At step 1, the spectra is smoothened by averaging each point measurement over a few points on either side of it using the equation (3) shown below.

$${S}_{smooth} ( {x}_{i} ) = \frac{{\sum }_{j = - m}^{m} S ( {x}_{i + j} )}{2 m + 1} \qquad ( 3 )$$

As shown above, m is the number of points in the smoothing window centered around xᵢ.

At step 2, a first order derivative and second order derivative are computed on the smoothed signal using the equation (4) shown below.

S′(xᵢ)=Sₛₘₒₒₜₕ(x_{i+gap})−S(x_{i-gap}) (4)

As shown above, gap is the distance between two peak values in the signal.

In an implementation, a plurality of parameters of the NW filter are updated, e.g., optimized, based on a correlation of consistent or global features, as explained in detail elsewhere herein.

Based on removing the noise, the processor 104 may obtain a noise removed NIR glucose data as a set of noise removed glucose spectra corresponding to plurality of reference glucose values.

The processor 104 may remove drift from the noise removed glucose data to obtain a preprocessed NIR glucose data. In an implementation, the drift is removed by applying Discrete Wavelet Transform (DWT) to the noise removed NIR glucose data. DWT analyzes the signals using mathematical functions referred to as wavelets. These functions divide the signal information into different frequency components, without any modification of signal shape, amplitude, and frequency components. As such, the wavelets are a windowing technique of variable dimension. Using greater time intervals, the information at low frequencies becomes more precise, and with smaller regions, the focus is posed in the information at high frequencies. The resulting mapping is of the scale-time form, being the frequency related to the scale. DWT is better understood after introducing continuous wavelet transform (CWT). The CWT analysis formula for a function ƒ(t) is given by equation (5) shown below.

W(s,τ)=∫ƒ(t)ψ*(s, τ) dt (5)

As shown above, ψ(s, τ) is the wavelet function given by equation (6) shown below.

$$Ψ ( s , τ ) = \frac{1}{\sqrt{s}} ψ ( \frac{t - τ}{s} ) \qquad ( 6 )$$

As shown in equations (5) and (6), s and τ are the scale and translation parameters, respectively. ψ(t) is referred to as the wavelet prototype function, also called as analyzing wavelet or mother wavelet. The wavelets are generated from the single basic wavelet, i.e., the wavelet prototype function, by scaling and translation as given by equation (6).

The CWT synthesis formula is given by equation (7) shown below.

$$f ( t ) = \frac{1}{{C}_{Ψ}} \int \int W ( s , τ ) \frac{1}{\sqrt{s}} ψ ( \frac{t - τ}{s} ) d τ \frac{ds}{{ds}^{2}} , {C}_{Ψ} = \int \vert ψ ( ω ) \vert d ω \qquad ( 7 )$$

The DWT uses a discretization of the scale and translation parameters s and τ as shown in equation (8) shown below.

$${ψ}_{m , n} ( t ) = \frac{1}{\sqrt{{s}^{m}}} ( \frac{t - {ns}^{j}}{{s}^{j}} ) \qquad ( 8 )$$

The dyadic discretization results in equations (9) and (1) shown below.

$$\begin{matrix}{ψ}_{m , n} ( t ) = {2}^{- \frac{m}{2}} ψ ( {2}^{- m} t - n ) & ( 9 ) \\ {φ}_{m , n} ( t ) = {2}^{- \frac{m}{2}} φ ( {2}^{- m} t - n ) & ( 10 )\end{matrix}$$

As shown above, ϕ(t) is the scaling function that captures lower frequencies (s>1).

Now, the Signal x(t) may be represented at level −3 as provided by equation (11) shown below.

$$\begin{matrix}x ( t ) = {A}_{1} + {D}_{1} = {A}_{2} + {D}_{2} + {D}_{1} = {A}_{3} + {D}_{3} + {D}_{2} + {D}_{1} = {A}_{3} + A \\ = {A}_{m} + {\sum }_{m = 1}^{3} {D}_{m}\end{matrix} \qquad ( 11 )$$

In general, the signal at level-M is represented by equation (12) shown below

x(t)=A_{M}+Σₘ₌₁^{M}Dₘ (12)

Here, A_{M} is the approximation of x(t) and Dₘ are the details of the signal x(t) at level-M given by equation (13) shown below.

A_{M}=Σ_{n=-∞}^{+∞}a_{M}(n)ϕ_{M,n}(t), D_{M}=Σ_{n=-∞}^{+∞}d_{M}(n)ψ_{M,n}(t) (13)

Here, a_{M} and d_{M} are approximation coefficients and detail coefficients at level-M, respectively, given by analysis equation (14) shown below.

a_{M}(n)=<x(t), ϕ_{M,n}(t)>, d_{M}(n)=<x(t),ψ_{M,n}(t)> (14)

The approximation A_{M} of x(t) at higher level-M captures low frequency components.

In accordance with an embodiment, the processor 104 may implement DWT to remove the drift by first estimating the drift and then removing the drift. The processor 104 may select an optimal wavelet function from a plurality of wavelet prototype functions as a wavelet function that exhibits maximum correlation with the plurality of reference glucose values. In an implementation, the processor 104 may select a wavelet prototype function from Wavelet Prototype Function library Ψ of N possible wavelets.

The processor 104 may scale each wavelet ψ^{k}(t) ∈ Ψ to obtain ψₛ^{k}(t). For a given subject's blood glucose vector y^{l}, the correlation coefficient of k^{th} wavelet with l^{th} glucose vector at scale is obtained as Rₖ^{l}(s) as shown in equation (15) below.

Rₖ^{l}(s)=<ψₛ^{k}(t), y^{l}> (15)

The metric of k^{th} wavelet for l^{th} glucose vector is obtained as the maximum correlation encountered over all scales. This is given by equation (16) shown below.

ρₖ^{l}=max{Rₖ^{l}(s)} ∀k, s, l (16)

If there are Nₗ subjects in total, the processor 104 may obtain the optimal wavelet function as ψ^{γ}(t), where the wavelet index γ ∈ {1, 2, . . . , N} is given by equation (17) shown below.

γ=argmax_{k∈{1, 2, . . . , N}} {Σₗ₌₁^{N}^{ₗ}(ρₖ^{l})²} (17)

Based on obtaining the optimal wavelet function, the processor 104 may obtain a global decomposition level. As such, the processor 104 may obtain a plurality of subject-specific decomposition levels as a level at which the correlation between the DWT approximation and linear approximation of the DWT approximation exceeds a pre-defined threshold. The processor 104 may then obtain the global decomposition level as the average of all subject-specific decomposition levels.

The processor 104 may determine the decomposition level by checking if the l-level approximation yₐₚₚᵣₒₓ^{k}(l) resembles a straight line (under linear drift assumption). The check for resemblance to straight line may be done by fitting a linear regression to the l-level approximation to obtain ŷₚₚᵣₒₓ^{k}(l).

The processor 104 may then obtain a measure of straight line fit by computing a correlation metric of straight line fit given by equation (18) shown below.

$${R}^{k} ( l ) = \frac{{Σ ( {y}_{approx}^{k} ( l ) - \overline{{y}_{approx}^{k} ( l )} )}^{2}}{{Σ ( {\hat{y}}_{approx}^{k} ( l ) - \overline{{y}_{approx}^{k} ( l )} )}^{2}} \qquad ( 18 )$$

The decomposition level at which the R^{k}(l), the correlation for subject k at level-l exceeds a predefined threshold T determines the decomposition-level l^{k} of subject k using equation (19) shown below.

l^{k}={l: R^{k}(l)>T} (19)

The processor 104 may then obtain the global decomposition level as an average of individual decomposition levels using equation (20) shown below.

$${L}_{g} = round ( \frac{1}{N} {\sum }_{k = 1}^{N} {l}^{k} ) \qquad ( 20 )$$

In an example, the global decomposition level of 10 is obtained when the correlation threshold is considered as R^{k}(l)>0.995.

Based on selecting the optimal wavelet function and obtaining the global decomposition level, the processor 104 may determine the drift present in the noise removed NIR glucose data as a DWT approximation at the global decomposition level. The processor 104 may then remove the drift from the noise removed NIR glucose data to obtain the preprocessed NIR glucose data. The NIR glucose data may be represented as a matrix S contains 129 features as columns, and may be represented as:

$$S = [ \begin{matrix}{x}_{0}^{0} {x}_{1}^{0} … … {x}_{128}^{0} \\ {x}_{0}^{1} {x}_{1}^{1} … … {x}_{128}^{1} \\ … . \\ {x}_{0}^{N - 1} {x}_{1}^{N - 1} … … {x}_{128}^{N - 1}\end{matrix} ] = [ {S}_{0} \vert {S}_{1} \vert … \vert {S}_{128} ]$$

As shown above, feature refers to the column of the NIR glucose data matrix S. The processor 104 may remove the drift from each feature by following the below steps.

At step 1, n^{th} feature Sₙ=S(: , n) is obtained.

At step 2, wavelet approximation of feature Sₙ at decomposition-level L_{g} is performed to obtain Ŝₙ.

At step 3, drift-free feature is obtained as Sₙ^{nd}=Sₙ−Ŝₙ

Based on obtaining the preprocessed NIR glucose data, the processor 104 may obtain a set of global features from the preprocessed NIR glucose data for non-invasive monitoring of the blood glucose of the subject. As such, the set of global features may be stored in the memory for non-invasive monitoring of the blood glucose. In addition, in an implementation, the system 100 may include various algorithms/techniques for predicting the blood glucose using the set of global features. In another implementation, the system 100 may provide the set of global features to a separate device/system for predicting the blood glucose using the set of global features. In another implementation, the system 100 may provide the set of global features to other units or modules of the mobile device or the wearable device for predicting the blood glucose using the set of global features.

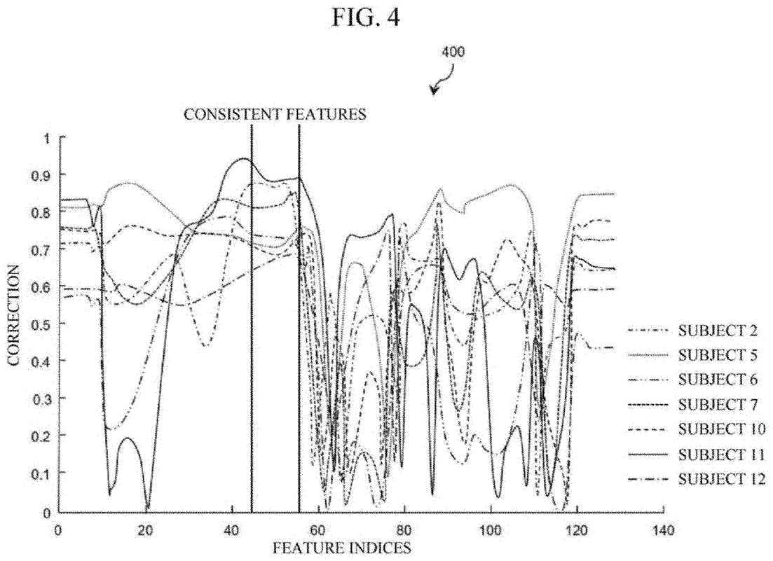

FIG. 4 illustrates an example graph 400 indicating the set of global features. The X-axis of the graph 400 represents feature indices and the Y-axis of the graph 400 represents correlation of a feature with the respective reference glucose values. Curves represented by various dashed lines correspond to subject 2, subject 5, subject 6, subject 7, subject 10, subject 11, and subject 12. In the example, the correlation values ≥0.68 are consistently observed for indices in the range 52-55 for all subjects. The feature indices 52, 53, 54 and 55 correspond to wavelength 1641 nm, 1643 nm, 1645 nm, and 1647 nm, respectively.

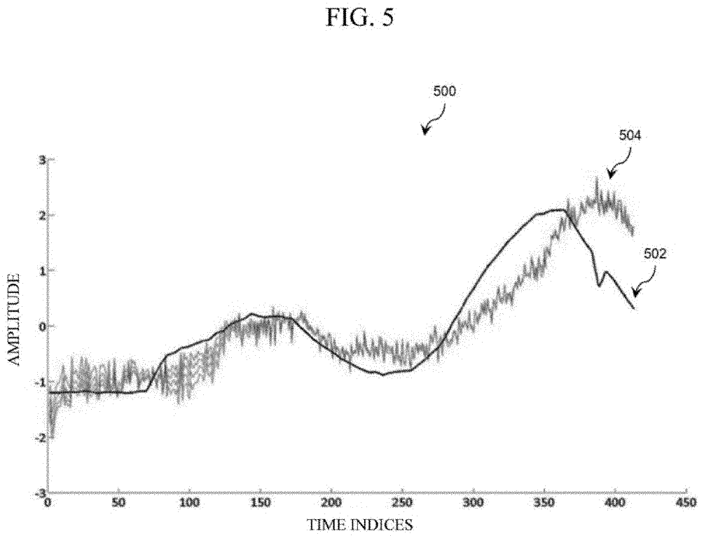

Accordingly, FIG. 5 illustrates an example graph 500 indicating a comparison between set of global features and predicted glucose value of subject 2. The X-axis of the graph 500 represents time and the Y-axis of the graph 500 represents the normalized amplitude of the reference glucose values and corresponding normalized features. Curve 502 represents the normalized reference glucose values of the subject 2. Curves 504 represent the four normalized global features 52, 53, 54 and 55. As can be observed from the graph 500, the curve 502 and the curves 504 are similar, indicating the performance of the preprocessing method.

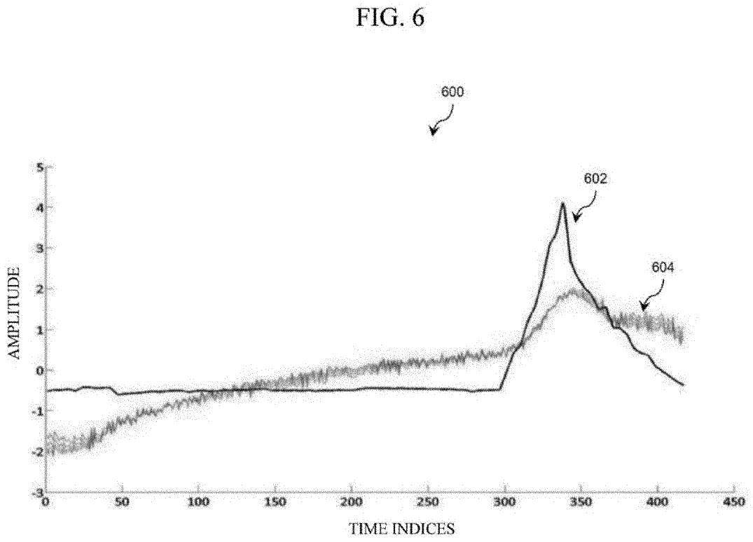

Accordingly, FIG. 6 illustrates an example graph 600 indicating a comparison between set of global features and predicted glucose of subject 10. The X-axis of the graph 600 represents feature indices and the Y-axis of the graph 600 represents the normalized amplitude of the reference glucose values and corresponding normalized features. Curve 602 represents the normalized reference glucose values of the subject 10. Curves 604 represent the four normalized global features 52, 53, 54 and 55. As can be observed from the graph 600, the curve 602 and the curves 604 are similar indicating the performance of the preprocessing method.

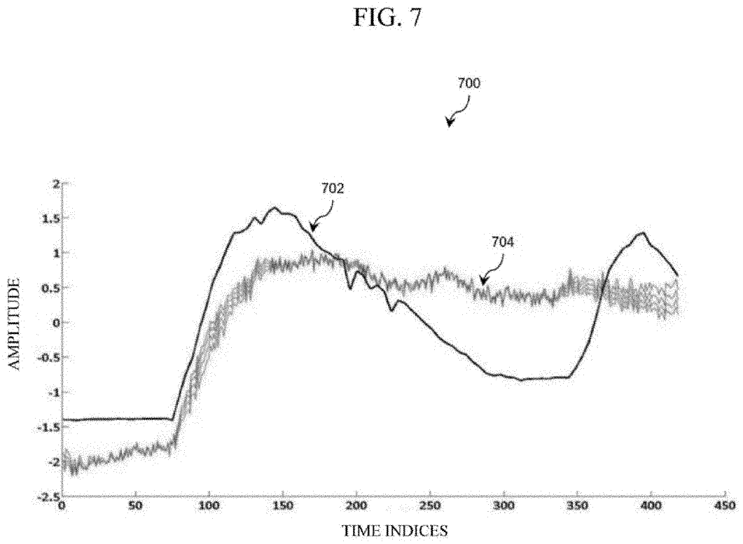

Accordingly, FIG. 7 illustrates an example graph 700 indicating a comparison between set of global features and predicted glucose of subject 6. The X-axis of the graph 700 represents feature indices and the Y-axis of the graph 700 represents the normalized amplitude of the reference glucose values and corresponding normalized features. Curve 702 represents the normalized reference glucose values of the subject 6. Curves 704 represent the four normalized global features 52, 53, 54 and 55. As can be observed from the graph 700, the curve 702 and the curves 704 are similar, indicating the performance of the preprocessing method.

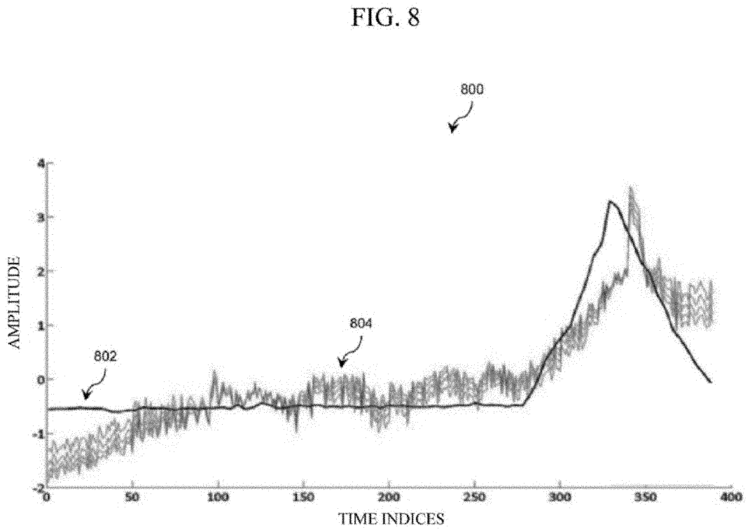

Accordingly, FIG. 8 illustrates an example graph 800 indicating a comparison between set of global features and predicted glucose of subject 7. The X-axis of the graph 800 represents feature indices and the Y-axis of the graph 800 represents the normalized amplitude of the reference glucose values and corresponding normalized features. Curve 802 represents the normalized reference glucose values of the subject 7. Curves 804 represent the four normalized global features 52, 53, 54 and 55. As can be observed from the graph 800, the curve 802 and the curves 804 are similar, indicating the performance of the preprocessing method.

Further, as described above, in an implementation the predefined spectral filter is a Norris-Williams filter. Accordingly, in such an implementation, the processor 104 updates the plurality of parameters of the Norris-Williams filter based on the set of global features. The plurality of parameters includes a gap of the Norris-Williams filter and a window size of the Norris-Williams filter. As such, the processor 104 may obtain an optimal value of the gap of the Norris-Williams filter from a predefined gap-set such that the optimal value of the gap provides highest correlation between the set of global features and the plurality of reference glucose values. Similarly, the processor 104 may obtain an optimal value of the window size of the Norris-Williams filter from a predefined window-size-set such that the optimal value of the window size provides highest correlation between the set of global features and the plurality of reference glucose values. The values of window size m and gap g are obtained by maximizing the quantity R_{i,j}^{(m,g)}. Here, R_{i,j}^{(m,g)} is the correlation of j^{th} global feature of i^{th} subject for a given values of m and g, given by the equation (21) shown below.

R_{i,j}^{(m,g)}=<fⱼⁱ, y^{j}> (21)

The optimal values mₒₚₜ, gₒₚₜ are given by the equation (22) shown below.

(mₒₚₜ, gₒₚₜ)=argmax_{a∈[1,m}_{ₘₐₓ}_{],b∈[1,g}_{ₘₐₓ}_{]}{R_{i,j}^{(m,g)}}, ∀i, j (22)

The updated plurality of parameters of the Norris-Williams filter can then be applied to another NIR spectroscopy data obtained from the subject for removal of noise. As such, the updated plurality of parameters may be stored in the memory for application at a future instance. Based on obtaining the glucose spectra from the another NIR spectroscopy data, the processor 104 may then remove noise from the glucose spectra by applying the updated plurality of parameters in a manner as described earlier.

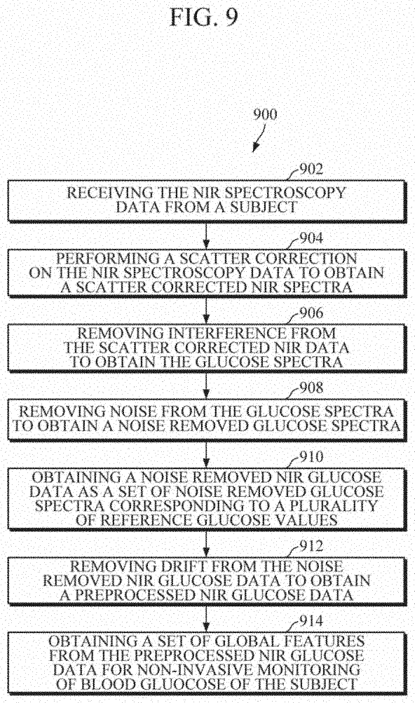

FIG. 9 illustrates a flow diagram of a method 900 for preprocessing near infrared (NIR) spectroscopy data for non-invasive monitoring of blood glucose, in accordance with the embodiment of the present disclosure. The method 900 may be implemented in the system 100 using components thereof, as described above. In an implementation, the method 900 may be executed by the sensor 102 and the processor 104. Further, for the sake of brevity, details of the present disclosure that are explained in detail in the description of FIGS. 1 through 8 are not reiterated in detail in the description of FIG. 9.

At operation 902, the method 900 includes receiving the NIR spectroscopy data from a subject. The NIR spectroscopy data comprises spectra of a plurality of interfering components and glucose spectra. For example, the sensor 102 may receive the NIR spectroscopy data from the subject.

At operation 904, the method 900 includes performing a scatter correction on the NIR spectroscopy data to obtain a scatter corrected NIR spectra. For example, the processor 104 may perform the scatter correction on the NIR spectroscopy data to obtain the scatter corrected NIR spectra.

At operation 906, the method 900 includes removing interference from the scatter corrected NIR data to obtain the glucose spectra. The step of removing interference includes applying Extended Multiplicative Scattering Correction (EMSC) to the scatter corrected NIR spectra to obtain the glucose spectra. For example, the processor 104 may remove interference from the scatter corrected NIR spectra to obtain the glucose spectra.

At operation 908, the method 900 includes removing noise from the glucose spectra to obtain a noise removed glucose spectra. The operation of removing noise includes applying predefined spectral filtering to the glucose spectra. For example, the processor 104 may then remove noise from the glucose spectra to obtain noise removed glucose spectra.

At operation 910, the method 900 includes obtaining a noise removed NIR glucose data as a set of noise removed glucose spectra corresponding to a plurality of reference glucose values. For example, the processor 104 may obtain a noise removed NIR glucose data as a set of noise removed glucose spectra corresponding to plurality of reference glucose values.

At operation 912, the method 900 includes removing drift from the noise removed NIR glucose data to obtain a preprocessed NIR glucose data. For example, the processor 104 may remove drift from the noise removed glucose data to obtain a preprocessed NIR glucose data.

At operation 914, the method 900 includes obtaining a set of global features from the preprocessed NIR glucose data for non-invasive monitoring of blood glucose of the subject. For example, the processor 104 may obtain the set of global features from the preprocessed NIR glucose data for non-invasive monitoring of blood glucose.

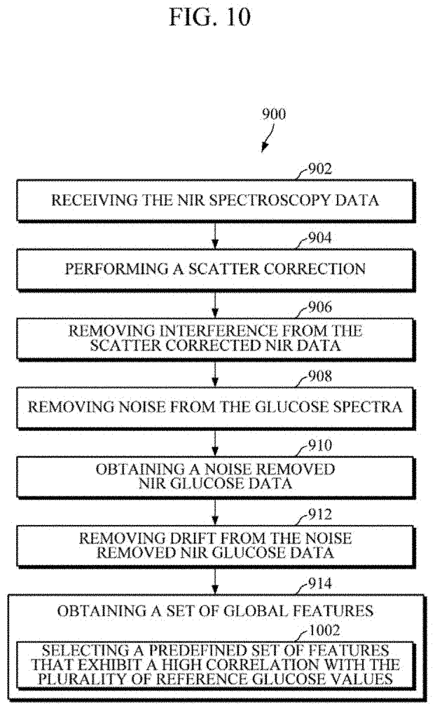

Further, the operation of obtaining the set of global features at operation 914 includes an additional operation. Referring to FIG. 10, at operation 1002, the method 900 includes selecting a predefined set of features that exhibit a high correlation with the plurality of reference glucose values. For example, the processor 104 may select a predefined set of features that exhibit a high correlation with the plurality of reference glucose values.

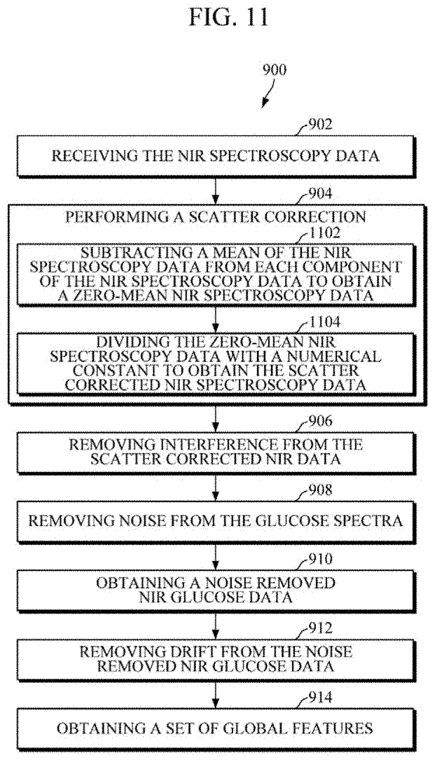

Further, the operation of performing the scatter correction at operation 904 includes further steps. Referring to FIG. 11, at operation 1102, the method 900 includes subtracting a mean of the NIR spectroscopy data from each component of the NIR spectroscopy data to obtain a zero-mean NIR spectroscopy data. At operation 1104, the method 900 includes dividing the zero-mean NIR spectroscopy data with a numerical constant to obtain the scatter corrected NIR spectroscopy data. For example, the processor 104 may obtain the zero-mean NIR spectroscopy data and then obtain the scatter corrected NIR spectroscopy.

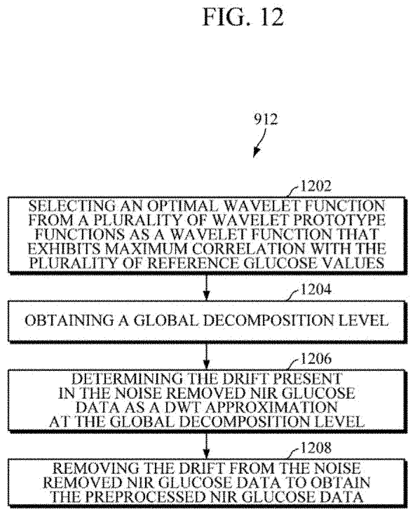

Further, the drift is removed at operation 912 by applying Discrete Wavelet Transform (DWT) to the noise removed NIR glucose data. As such, the operation of removing the drift at operation 910 comprises further steps. Referring to FIG. 12, at operation 1202, the method 900 includes selecting an optimal wavelet function from a plurality of wavelet prototype functions as a wavelet function that exhibits maximum correlation with the plurality of reference glucose values. At operation 1204, the method 900 includes obtaining a global decomposition level. At operation 1206, the method 900 includes determining the drift present in the noise removed NIR glucose data as a DWT approximation at the global decomposition level. At operation 1208, the method 900 includes removing the drift from the noise removed NIR glucose data to obtain the preprocessed NIR glucose data. For example, the processor 104 may remove drift from the noise removed glucose data by applying DWT.

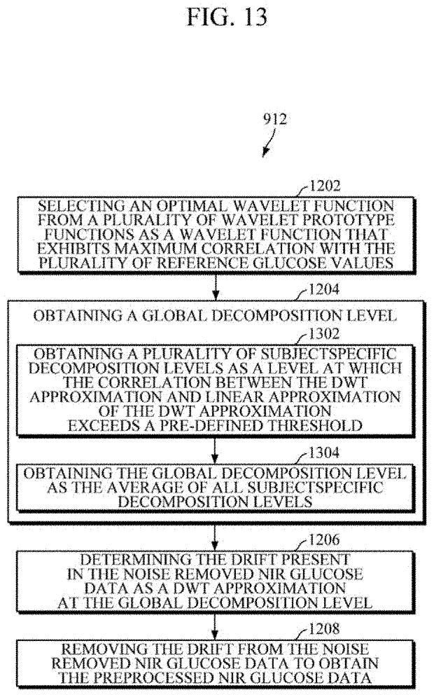

Further, the operation of obtaining the global decomposition level at operation 1204 comprises further operations. As such, referring to FIG. 13, at operation 1302, the method at operation 1204 includes obtaining a plurality of subject-specific decomposition levels as a level at which the correlation between the DWT approximation and linear approximation of the DWT approximation exceeds a pre-defined threshold. At operation 1304, the method at operation 1204 includes obtaining the global decomposition level as the average of all subject-specific decomposition levels. For example, the processor 104 may obtain the global decomposition level.

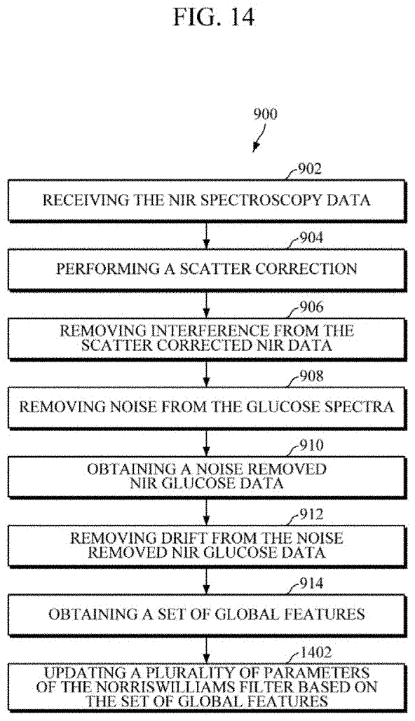

Further, the predefined spectral filter for removing noise at operation 908 is a Norris-Williams filter. As such, referring to FIG. 14, at operation 1402, the method 900 includes updating a plurality of parameters of the Norris-Williams filter based on the set of global features. The plurality of parameters includes a gap of the Norris-Williams filter and a window size of the Norris-Williams filter. For example, the processor 104 may update the plurality of parameters of the Norris-Williams filter based on the set of global features.

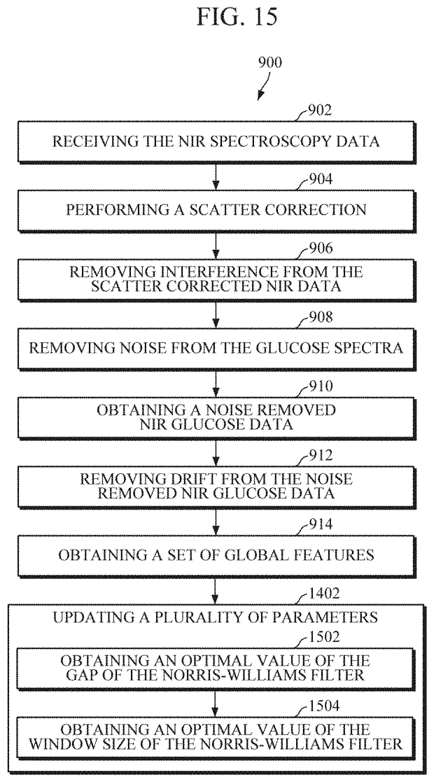

Further, the operation of updating parameters at operation 1402 comprises further steps. Referring to FIG. 15, at operation 1502, the method 900 includes obtaining an optimal value of the gap of the Norris-Williams filter from a predefined gap-set such that the optimal value of the gap provides highest correlation between the set of global features and the plurality of reference glucose values. At operation 1504, the method 900 includes obtaining an optimal value of the window size of the Norris-Williams filter from a predefined window-size-set such that the optimal value of the window size provides highest correlation between the set of global features and the plurality of reference glucose values. For example, the processor 104 may obtain optimal values of the gap of the Norris-Williams filter and the window size of the Norris-Williams filter.

Thus, the present disclosure enables preprocessing of the NIR spectroscopy data such that the effects of noise and drift are removed from the NIR spectroscopy data prior to predicting blood glucose levels. Further, the preprocessing of the NIR spectroscopy data includes scatter correction and noise removal along with drift correction to obtain high quality data. The improvement in the quality of the data results in better prediction accuracy of the blood glucose value using standard machine learning methods. Further, the preprocessing of the NIR spectroscopy data obtains global features that exhibit high correlation with the reference glucose values and are therefore universal, i.e., the features are common across all test subjects. As such, accurate prediction of blood glucose levels is obtained for any subject's bio-profile.

While specific language has been used to describe the present disclosure, any limitations arising on account thereto, are not intended. As would be apparent to a person skilled in the art, various working modifications may be made to the method in order to implement the inventive concept as taught herein. The drawings and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. Clearly, the present disclosure may be otherwise variously embodied, and practiced within the scope of the following claims.

What is claimed is:

1. A method for preprocessing near infrared (NIR) spectroscopy data for non-invasive monitoring of blood glucose, the method comprising:
receiving the NIR spectroscopy data from a subject;
performing a scatter correction on the NIR spectroscopy data to obtain scatter corrected NIR spectra;
removing interference from the scatter corrected NIR spectra to obtain glucose spectra;
removing noise from the glucose spectra to obtain noise removed glucose spectra;
obtaining noise removed NIR glucose data as a set of noise removed glucose spectra corresponding to a plurality of reference glucose values;
removing drift from the noise removed NIR glucose data to obtain preprocessed NIR glucose data; and
obtaining a set of global features from the preprocessed NIR glucose data for non-invasive monitoring of blood glucose of the subject.

2. The method as claimed in claim 1, wherein the NIR spectroscopy data comprises spectra of a plurality of interfering components and the glucose spectra.

3. The method as claimed in claim 1, wherein obtaining the set of global features comprises selecting a predefined set of features that exhibit a high correlation with the plurality of reference glucose values.

4. The method as claimed in claim 1, wherein removing the interference comprises applying Extended Multiplicative Scattering Correction (EMSC) to the scatter corrected NIR spectra to obtain the glucose spectra.

5. The method as claimed in claim 1, wherein performing the scatter correction on the NIR spectroscopy data comprises:
subtracting a mean of the NIR spectroscopy data from each component of the NIR spectroscopy data to obtain a zero-mean NIR spectroscopy data; and
dividing the zero-mean NIR spectroscopy data with a numerical constant to obtain the scatter corrected NIR spectroscopy data.

6. The method as claimed in claim 1, wherein the drift is removed by applying Discrete Wavelet Transform (DWT) to the noise removed NIR glucose data.

7. The method as claimed in claim 6, wherein removing the drift from the noise removed glucose data using DWT comprises:
selecting an optimal wavelet function from a plurality of wavelet prototype functions, wherein the optimal wavelet function is a wavelet function that exhibits maximum correlation with the plurality of reference glucose values;
obtaining a global decomposition level;
determining the drift present in the noise removed NIR glucose data as a DWT approximation at the global decomposition level; and
removing the drift from the noise removed NIR glucose data to obtain the preprocessed NIR glucose data.

8. The method as claimed in claim 7, wherein obtaining the global decomposition level comprises:
obtaining a plurality of subject-specific decomposition levels as a level at which the correlation between the DWT approximation and linear approximation of the DWT approximation exceeds a pre-defined threshold; and
obtaining the global decomposition level as an average of all subject-specific decomposition levels.

9. The method as claimed in claim 1, wherein removing the noise comprises applying a predefined spectral filter to the glucose spectra to obtain the noise removed glucose spectra.

10. The method as claimed in claim 9, wherein the predefined spectral filter is a Norris-Williams filter.

11. The method as claimed in claim 10, further comprising:
updating a plurality of parameters of the Norris-Williams filter based on the set of global features, the plurality of parameters including a gap of the Norris-Williams filter and a window size of the Norris-Williams filter.

12. The method as claimed in claim 11, wherein updating the parameters of the Norris-Williams filter comprises:
obtaining an optimal value of the gap of the Norris-Williams filter from a predefined gap-set such that the optimal value of the gap provides highest correlation between the set of global features and the plurality of reference glucose values; and
obtaining an optimal value of the window size of the Norris-Williams filter from a predefined window-size-set such that the optimal value of the window size provides highest correlation between the set of global features and the plurality of reference glucose values.

13. A system for preprocessing near infrared (NIR) spectroscopy data for non-invasive monitoring of blood glucose, the system comprising:
a memory configured to store instructions; and
a processor configured to execute the instructions to:
perform a scatter correction on NIR spectroscopy data from a subject to obtain scatter corrected NIR spectra;
remove interference from the scatter corrected NIR spectra to obtain glucose spectra;
remove noise from the glucose spectra to obtain noise removed glucose spectra;
obtain noise removed NIR glucose data as a set of noise removed glucose spectra corresponding to plurality of reference glucose values;
remove drift from the noise removed glucose spectra to obtain preprocessed NIR glucose data; and
obtain a set of global features from the preprocessed NIR glucose data for non-invasive monitoring of the blood glucose of the subject.

14. The system as claimed in claim 13, wherein the NIR spectroscopy data comprises spectra of a plurality of interfering components and the glucose spectra.

15. The system as claimed in claim 13, wherein to obtain the set of global features the processor is configured to select a predefined set of features that exhibit a high correlation with the plurality of reference glucose values.

16. The system as claimed in claim 13, wherein to remove the interference the processor is configured to apply Extended Multiplicative scattering correction (EMSC) to the scatter corrected NIR spectra to obtain the glucose spectra.

17. The system as claimed in claim 13, wherein to perform the scatter correction the processor is configured to:
subtract a mean of the NIR spectroscopy data from each component of the NIR spectroscopy data to obtain zero-mean NIR spectroscopy data; and
divide the zero-mean NIR spectroscopy data with a numerical constant to obtain the scatter corrected NIR spectroscopy data.

18. The system as claimed in claim 13, wherein the drift is removed by applying Discrete Wavelet Transform (DWT) to the noise removed NIR glucose data.

19. The system as claimed in claim 18, wherein to remove the drift the processor is configured to:
select an optimal wavelet function from a plurality of wavelet prototype functions, the optimal wavelet function is a wavelet function that exhibits maximum correlation with the plurality of reference glucose values;
obtain a global decomposition level;
determine the drift present in the noise removed NIR glucose data as a DWT approximation at the global decomposition level; and
remove the drift from the noise removed NIR glucose data to obtain the preprocessed NIR glucose data.

20. The system as claimed in claim 19, wherein to obtain the global decomposition level the processor is configured to:
obtain a plurality of subject-specific decomposition levels as a level at which the correlation between the DWT approximation and linear approximation of the DWT approximation exceeds a pre-defined threshold; and
obtain the global decomposition level as an average of the plurality of subject-specific decomposition levels.

21. The system as claimed in claim 13, wherein to removing noise, the processor is configured to apply a predefined spectral filter to the glucose spectra to obtain the noise removed glucose spectra.

22. The system as claimed in claim 21, wherein the predefined filter is a Norris-Williams filter.

23. The system as claimed in claim 22, wherein the processor is configured to:
update a plurality of parameters of the Norris-Williams filter based on the set of global features, the plurality of parameters including a gap of the Norris-Williams filter and a window size of the Norris-Williams filter.

24. The system as claimed in claim 23, wherein to update the parameter of the Norris-Williams filter the processor is configured to:
obtain an optimal value of the gap of the Norris-Williams filter from a predefined gap-set such that the optimal value of the gap provides highest correlation between the set of global features and the plurality of reference glucose values; and
obtain an optimal value of the window size of the Norris-Williams filter from a predefined window-size-set such that the optimal value of the window size provides highest correlation between the set of global features and the plurality of reference glucose values.
